(19) United States
(12) Patent Application Publication
(10) Pub. No.: US 2024/0076705 A1
(43) Pub. Date: Mar. 7, 2024

(54) LAMBDA-CARRAGEENASE MUTANT OUC-CGLA-DPQQ AND APPLICATION THEREOF

(71) Applicant: Ocean University of China, Qingdao (CN)

(72) Inventors: Xiangzhao MAO, Qingdao (CN); Hong JIANG, Qingdao (CN); Zewei LU, Qingdao (CN); Yueyang DONG, Qingdao (CN); Chengqiang LI, Qingdao (CN)

(21) Appl. No.: 18/186,488

(22) Filed: Mar. 20, 2023

(30) Foreign Application Priority Data
Aug. 31, 2022 (CN) 202211050436.2

(51) Int. Cl. C12P 19/04; C12N 9/24

(52) U.S. Cl. CPC C12P 19/04; C12N 9/2402; C12Y 302/01162

(57) ABSTRACT

The present disclosure discloses λ-carrageenase mutant OUC-CglA-DPQQ, which has an amino acid sequence as set forth in SEQ ID NO. 1 and an encoding gene as set forth in SEQ ID NO. 2. The λ-carrageenase mutant OUC-CglA-DPQQ is applied to degradation of carrageenan/preparation of λ-carrageenan oligosaccharides with high polymerization degree. The present disclosure further discloses a method for degrading carrageenan/preparing λ-carrageenan oligosaccharides with high polymerization degree by using the λ-carrageenase mutant OUC-CglA-DPQQ. The λ-carrageenase mutant OUC-CglA-DPQQ of the present disclosure can act on low viscosity carrageenan, and the polymerization degree of final product λ-carrageenan oligosaccharides is 10-20. The λ-carrageenase mutant of the present disclosure is excellent in enzymatic properties and specificity, and has important industrial application value and economic value in the preparation of λ-carrageenan oligosaccharides via an enzyme method.

### REFERENCE TO AN ELECTRONIC SEQUENCE LISTING

The content of the electronic sequence listing (2023-03-20_SequenceListing.xml; Size: 7,380 bytes; and Date of Creation: Mar. 20, 2023) is herein incorporated by reference in its entirety.

### TECHNICAL FIELD

The present disclosure relates to λ-carrageenase mutant OUC-CglA-DPQQ and application thereof, belonging to the technical field of functional enzymes.

### BACKGROUND ART

Carrageenan is an acidic polysaccharide extracted from red algae, which has repetitive α-1, 4-D-galactopyranose and β-1, 3-D-galactopyranose (or 3,6-anhydro-D-galactopyranose) disaccharide unit backbone structure. Carrageenan oligosaccharides are the degradation products of the carrageenan. According to the different binding forms of sulfate, the carrageenan oligosaccharides can be divided into ten different types of idealized repetitive disaccharides, which are α-, β-, λ-, δ-, κ-, μ-, θ-, δ-, I-, and V-carrageenan oligosaccharides, with differences mainly in degree of sulfation and the presence or absence of 3,6-anhydrogalactose residues.

λ-carrageenan oligosaccharides have anticoagulant, antithrombotic, anti-inflammatory, antiviral, antitumor and other effects. In recent years, studies have confirmed that the λ-carrageenan oligosaccharides also play an important role in the antioxidation and free radical scavenging of organisms. Therefore, how to efficiently prepare the λ-carrageenan oligosaccharides has increasingly become a hot topic of research.

The methods for preparing the λ-carrageenan oligosaccharides include physical methods, chemical methods and enzymatic hydrolysis methods. The physical methods mainly refer to methods for degrading λ-carrageenan by physical means such as ultrasound and microwave. The chemical methods mainly include acid hydrolysis and oxidation degradation. The most common degradation methods are the acid hydrolysis and enzymatic hydrolysis. Compared with the acid hydrolysis, the enzymatic hydrolysis has the advantages of mild reaction, single product, easy separation, and the like. However, there are few records about λ-carrageenase in the literature, and most of the product components are oligosaccharides with low polymerization degree. Therefore, it is of a great significance to explore new λ-carrageenase and construct new engineering bacteria.

### SUMMARY OF THE INVENTION

In view of the above-mentioned prior art, the present disclosure provides a novel degrading enzyme, i.e., λ-carrageenase mutant OUC-CglA-DPQQ, which can degrade carrageenan to produce λ-carrageenan oligosaccharides, so that the oligosaccharides with high polymerization degree can be prepared by degradation. The present disclosure further provides application of the mutant enzyme, and a method for degrading carrageenan/preparing λ-carrageenan oligosaccharides.

The present disclosure is realized by the following technical solutions:

Provided is λ-carrageenase mutant OUC-CglA-DPQQ, which has an amino acid sequence as set forth in SEQ ID NO. 1.

The amino acid sequence (SEQ ID NO. 1) of the λ-carrageenase mutant OUC-CglA-DPQQ is as follows:

MRENNAPMYAVTVITKDKTPYIVCGGYDKNIYYLSSSGDLLKTIASKDY
SVEKAKGANSKEVPTGNAHISNFIRTIKKADGSEVLAVQGVNHTMSIQA
RGSLYLFHPLDEKPFKTIDLEGKRLLGELKVVDVGLDGDQEIIAGSSTM
IHESSLLKVDLETYEQELFEISKIKKKIDRFGYRVTQPEIITQNGKQSY
FVLFGSRILLVPLDFNLEKTEVLVSKYSFNDMWKDGKGNIILASIQSGG
SEIYVLNPNKPNWKQAYENLIPKGKIASIIANTKEVKEQLNVFTAPIEE
RKPLPVYLMSEGVPPSIKTLVDDIKENYKSPVFLNRSGSDKEKWDRSSI
ENEKYRDRRDRRMKYILSQDQVIKKITSKYKGGPGIAYWGGHGNDPYMY
QLSTTKKVLNAAKGKKTVLIFPELEDHSDNFNYVMEDYFYPLAKHARET
NANIYVRTKHAFWQANVYLPTWSRLVSGEFSDVFVPAMEETTDKSMDLS
IAGRLGIWTSGATDSWGSRCARDNPSLDRLRQHSHQMLPNHFLRNMIYH
VSSGAQYLDNFNVDQEYMSLLWELIAKGALYVPKRSEILSFSPVNLGML
SPDKHYLEESSNAKWTTFYDEEKEKNNPLVFSHLNGSWPGAPVTEWDFS
KYAGGVQDRRLNFLPTYTNGLVLLTPPQNGIYADKKAKRGTLTSHLHPM
YKNIMKEYYTDGRNYYSADGTQTYKANEYYKVIAKDIKDNAKKLPLTVS
GDVAWVVAQSSPKHLRLTLIDGGYLNPDDREVTIQFNTVVPIKVTDLLS
GEEFTPNANGELQIKVSCGLFRFIDIEFTGFKLE.

Provided is an encoding gene of the λ-carrageenase mutant OUC-CglA-DPQQ, which has a nucleotide sequence as set forth in SEQ ID NO. 2.

The nucleotide sequence (SEQ ID NO. 2) of the encoding gene of the λ-carrageenase mutant OUC-CglA-DPQQ is as follows:

5′-ATGCGCGAAAACAACGCGCCAATGTACGCAGTAACCGTTATTACCA
AAGATAAAACCCCGTACATTGTATGCGGTGGTTATGACAAAAATATCTA
CTACCTGTCTAGTTCCGGCGATCTGCTGAAAACCATTGCGAGCAAAGAC
TATTCGGTTGAAAAAGCAAAAGGCGCGAACAGCAAAGAAGTGCCGACTG
GCAACGCCCACATTTCCAACTTCATTCGCACCATCAAAAAAGCAGACGG
TTCTGAAGTGCTGGCGGTTCAGGGCGTGAACCACACCATGTCCATCCAA
GCTCGCGGTAGTCTGTACCTGTTCCATCCGCTGGACGAAAAACCGTTTA
AAACCATTGATCTGGAAGGCAAACGTCTGCTGGGCGAACTGAAGGTGGT
TGATGTTGGCCTGGACGGCGACCAGGAAATTATCGCGGGTTCTTCCACC
ATGATCCACGAATCTTCTCTGCTGAAAGTTGATCTGGAAACCTACGAAC
AGGAACTGTTCGAAATCAGCAAAATTAAAAAGAAAATCGATCGCTTCGG
CTATCGCGTGACCCAGCCGGAAATCATTACTCAGAACGGTAAACAGTCT
TACTTCGTGCTGTTCGGCAGCCGTATCCTGCTGGTGCCGCTGGATTTTA
ACCTCGAAAAAACCGAAGTTCTGGTTAGCAAATACAGCTTCAATGACAT
GTGGAAAGATGGCAAAGGCAATATCATCTTGGCGAGCATCCAGAGCGGT
GGCTCTGAAATCTACGTTCTGAACCCGAACAAACCAAACTGGAAACAGG
CGTACGAAAACCTGATCCCGAAAGGCAAAATCGCGAGCATTATCGCCAA
TACCAAAGAAGTTAAAGAACAGCTGAACGTTTTTACCGCGCCGATCGAA
GAACGCAAACCGCTGCCGGTTTACCTGATGTCTGAGGGTGTGCCGCCGA
GCATCAAAACGCTGGTTGATGATATCAAAGAAAACTACAAATCTCCGGT
TTTCCTGAACCGCAGCGGTTCTGACAAAGAAAAATGGGATCGCTCCTCT
ATCGAAAACGAAAAATATCGTGATCGCCGCGACCGCCGTATGAAATACA
TTCTGTCTCAGGATCAGGTCATCAAGAAAATCACATCGAAATACAAAGG
CGGTCCGGGCATCGCCTACTGGGGCGGTCACGGCAACGATCCGTACATG
TACCAGCTGTCTACCACTAAGAAAGTGCTGAACGCTGCGAAAGGCAAGA
AAACTGTGCTGATTTTCCCGGAATTGGAAGATCACTCTGATAATTTCAA
CTACGTTATGGAAGATTACTTCTACCCGCTGGCCAAACACGCTCGTGAA
ACCAACGCCAACATCTATGTTCGTACCAAGCACGCGTTTTGGCAGGCAA
ATGTGTATTTGCCGACCTGGTCCCGTCTGGTTTCGGGCGAGTTTTCTGA
TGTGTTTGTTCCGGCAATGGAAGAAACCACCGACAAGAGCATGGACCTG
AGCATCGCGGGCCGTCTGGGCATTTGGACCAGCGGCGCTACCGACTCCT
GGGGCAGCCGTTGCGCACGTGATAACCCGAGCCTGGATCGTCTGCGCCA
GCATAGCCACCAGATGCTGCCGAACCACTTCCTGCGCAACATGATTTAC
CATGTTAGCTCCGGTGCGCAGTACCTGGATAACTTCAACGTGGACCAGG
AATACATGAGCCTGCTGTGGGAACTGATTGCTAAAGGTGCACTGTACGT
TCCGAAACGTAGTGAAATTCTGTCCTTCTCTCCGGTTAACCTGGGTATG
CTGAGCCCGGATAAACACTACCTGGAGGAAAGCAGTAACGCGAAGTGGA
CCACCTTCTACGATGAAGAAAAAGAGAAAAACAACCCTCTGGTCTTTAG
CCACCTGAACGGTTCTTGGCCGGGCGCACCCGTGACCGAATGGGATTTT
TCTAAATATGCGGGTGGCGTGCAGGATCGTCGTCTGAACTTCCTGCCGA
CCTATACTAACGGCCTGGTACTGCTGACTCCGCCGCAGAACGGTATCTA
CGCTGATAAAAAAGCGAAACGTGGTACCCTGACTAGCCACCTGCACCCG
ATGTACAAAAACATTATGAAGGAATATTACACCGATGGCCGTAATTACT
ACAGCGCGGACGGCACCCAGACCTACAAAGCGAATGAATACTATAAAGT
GATCGCAAAAGATATTAAAGATAACGCGAAAAAACTGCCGCTGACCGTT
AGCGGTGACGTGGCGTGGGTTGTTGCGCAGAGCTCCCCGAAACATCTGC
GTCTGACCCTGATCGACGGCGGCTACCTGAACCCGGACGACCGTGAAGT
GACCATCCAGTTCAACACCGTGGTTCCGATTAAAGTTACTGACCTGCTG
AGCGGTGAAGAATTCACTCCGAACGCTAACGGTGAACTGCAGATTAAAG
TTAGTTGCGGCCTGTTCCGTTTCATTGACATCGAATTCACCGGCTTCAA
ACTCGAG-3'.

Provided is application of the λ-carrageenase mutant OUC-CglA-DPQQ to degradation of carrageenan/preparation of λ-carrageenan oligosaccharides. The λ-carrageenan oligosaccharides have high polymerization degree (10-20).

Provided is a method for degrading the carrageenan/preparing the λ-carrageenan oligosaccharides. The λ-carrageenase mutant OUC-CglA-DPQQ is used to degrade the carrageenan to obtain the λ-carrageenan oligosaccharides with high polymerization degree (10-20), including any one or more of long-chain oligosaccharides such as decasaccharide, dodecasaccharide, tetradecasaccharide, hexadecasaccharide, octadecasaccharide and icosaccharide.

Further, the method for preparing λ-carrageenan oligosaccharides specifically includes the steps of: adding the λ-carrageenase mutant OUC-CglA-DPQQ into a λ-carrageenan solution, and degrading at 10-25° C., pH 6.0-8.0, to obtain a degradation product, that is, the λ-carrageenan oligosaccharides with high polymerization degree.

Further, the concentration of λ-carrageenan in the λ-carrageenan solution is 2-5 mg/ml, preferably 3 mg/ml.

Further, the temperature for degradation is 15° C.

Further, the pH for degradation is 7.0.

Further, the time for degradation is 1-12 hours, preferably 4-6 hours, and more preferably 2 hours.

Further, the λ-carrageenase mutant OUC-CglA-DPQQ is added into the λ-carrageenan solution in the form of an enzyme solution, and the enzyme dosage is 0.20-0.25 U, preferably 1.5 U.

Provided is an encoding gene of λ-carrageenase mutant OUC-CglA-DPQQ to preparation of λ-carrageenase mutant OUC-CglA-DPQQ.

Provided is a recombinant expression vector, which carries the encoding gene of λ-carrageenase mutant OUC-CglA-DPQQ.

Provided is a recombinant engineering bacterium, which has the encoding gene of λ-carrageenase mutant OUC-CglA-DPQQ inserted into a genome thereof so as to be capable of expressing the λ-carrageenase mutant OUC-CglA-DPQQ.

Provided is application of the recombinant expression vector or the recombinant engineering bacterium to preparation of λ-carrageenase mutant OUC-CglA-DPQQ.

Provided is an enzyme preparation, which includes the λ-carrageenase mutant OUC-CglA-DPQQ.

Provided is application of the enzyme preparation to degradation of carrageenan/the preparation of λ-carrageenan oligosaccharides.

The λ-carrageenase mutant OUC-CglA-DPQQ of the present disclosure has a specific enzyme activity of up to 51.59 U/mg at 15° C. and pH 7. The λ-carrageenase mutant OUC-CglA-DPQQ can act on a low viscosity λ-carrageenan substrate, and the polymerization degree of the final product λ-carrageenan oligosaccharides is 10-20. The present disclosure constructs the recombinant vector containing the encoding gene of λ-carrageenase mutant, realizes the heterologous expression in Escherichia coli, and provides a good basis for the industrial production and application of the enzyme. The λ-carrageenase mutant of the present disclosure is mild in reaction conditions, has excellent enzymatic properties and specificity, has a superior degradation effect on λ-carrageenan, degrades the λ-carrageenan into the long-chain oligosaccharides such as decasaccharide and dodecasaccharide, can be applied to preparation of antibacterial agents, antiviral agents, immunomodulators, antioxidants, and the like, and has important industrial application value and economic value in the preparation of λ-carrageenan oligosaccharides via an enzyme method.

Various terms and phrases used herein have their ordinary meanings as known to those skilled in the art.

### BRIEF DESCRIPTION OF THE DRAWINGS

FIG. 1: An SDS-PAGE electrophoregram of λ-carrageenase mutant, where M is standard protein Marker; 1 is crude enzyme protein; 2 is crude enzyme penetrating fluid protein; and 3 is purified concentrated λ-carrageenase mutant protein.

FIG. 2: A schematic diagram of the effect of temperature change on relative enzyme activity.

FIG. 3: A schematic diagram of the effect of pH change on relative enzyme activity.

FIG. 4: A liquid phase diagram of an enzymatic hydrolysate of the λ-carrageenase mutant.

FIG. 5: A mass spectrum of long-chain oligosaccharides, i.e., the enzymatic hydrolysate of the λ-carrageenase mutant.

FIG. 6: A comparison diagram of contents of purified enzyme protein before and after modification.

### DETAILED DESCRIPTION OF THE INVENTION

The present disclosure will be further described below in conjunction with examples. However, the scope of the present disclosure is not limited to the following examples. It will be understood by those skilled in the art that various changes and modifications can be made in the present disclosure without departing from the spirit and scope of the present disclosure.

The instruments, reagents, and materials involved in the following examples, unless otherwise specified, are all conventional instruments, reagents, and materials existing in the prior art, and can be obtained through formal commercial channels. The experimental methods, detection methods, and the like involved in the following examples, unless otherwise specified, are all conventional experimental methods and detection methods existing in the prior art.

### Example 1 Cloning of an Encoding Gene of λ-Carrageenase Mutant OUC-CglA-DPQQ

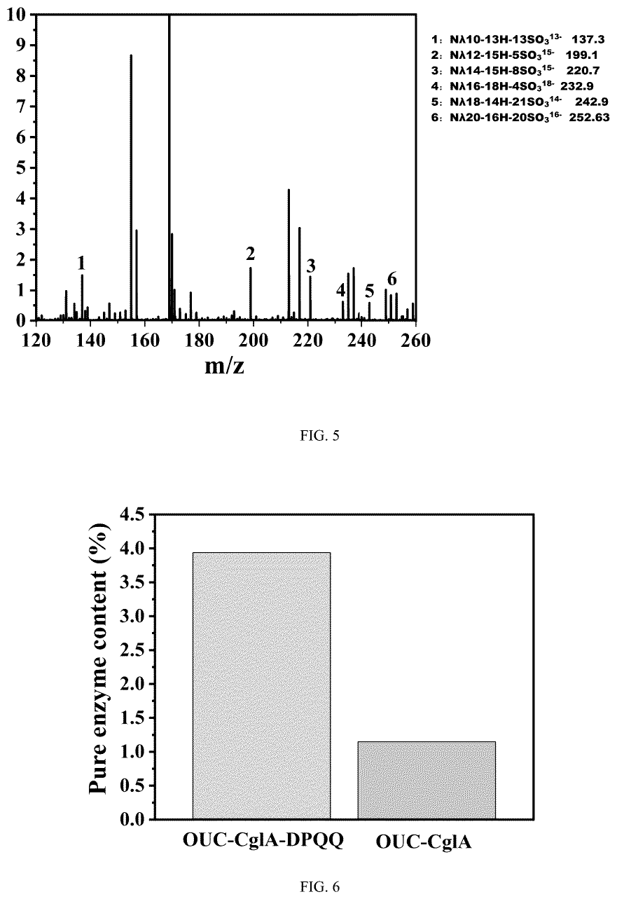

The inventor of the present disclosure compared the known λ-carrageenase with higher activity from a gene library of the NCBI, and successfully explored λ-carrageenase gene OUC-CglA (with a sequence number of WP_121067947.1) derived from Maribacter vaceletii. It was noticed that the domains of the enzyme contained a PQQ-like domain, and in a molecular docking model of enzyme protein and substrate, the domain might hinder the binding of an enzyme catalytic site and the substrate, so that it was speculated that the domain might have a certain effect on the activity of λ-carrageenase. Therefore, the gene was modified, and 118 amino acids in the PQQ-like domain part of the enzyme were deleted. After the modification, inclusion bodies formed when the gene was expressed in E. coli were fewer. After calculation, the content of the enzyme protein obtained by fermentation after the modification increased by 3 to 4 times, as shown in FIG. 6. The modified gene contains 2454 bases (as set forth in SEQ ID NO. 2), encodes 818 amino acids (as set forth in SEQ ID NO. 1), and is named λ-carrageenase mutant OUC-CglA-DPQQ.

A gene fragment of the OUC-CglA-DPQQ was artificially synthesized and used as a template for PCR amplification to obtain the gene fragment set forth in SEQ ID NO. 2. Specific primers used for amplification are as set forth in SEQ ID NO. 3 and 4.

Upstream primer: 5′-AGATATACCATGCGCGAAAACAACGCGCCA-3′, as set forth in SEQ ID NO. 3.

Downstream primer: 5′-GCGTTGTTTTCGCGCATGGTATATCTCCTTC-3′, as set forth in SEQ ID NO. 4.

A PCR reaction system was as follows: 2×PCR Buffer: 25 μl, dNTP: 10 μl, each of the primers: 1.5 μl, template: 1 μl, KOD Fx enzyme: 1 μl, sterile water: 10 μl, and the total system: 50 μl.

The PCR reaction conditions were: pre-denaturation at 94° C. for 5 min, denaturation at 95° C. for 20 s, annealing at 60° C. for 30 s, extension at 72° C. for 60 s, reaction for 30 cycles, and extension at 72° C. for 10 min.

7.681 Kb (all DNA of the whole plasmid of the mutant OUC-CglA-DPQQ obtained by a designed primer PCR) of PCR product fragment was recovered after agarose gel electrophoresis.

### Example 2 Construction of an Expression Vector Containing the Encoding Gene of λ-Carrageenase Mutant

The above amplified gene fragment is ligated with a pET-28a cloning vector by a seamless cloning technology, and the ligated product is transferred into E. coli DH5 a competent cells, coated on an LB medium solid plate (containing 50 μg/mL kanamycin), and incubated in an incubator at 37° C. for 16 hours; after that, a single clone was picked to an LB liquid medium containing 50 μg/mL kanamycin, and cultured for 12 hours in a 37° C. shaking table at 220 rpm. The single clone was sequenced after positive validation and named pET28a-OUC-CglA-DPQQ. The plasmid was stored at −20° C. for later use.

### Example 3 Construction of a Recombinant Engineering Bacterium

The plasmid extracted in Example 2 was transformed into E. coli BL21 competent cells of a host, and the constructed engineering bacterium grew on a kanamycin sulfate-resistant plate to obtain a recombinant expression strain.

### Example 4 Preparation of λ-Carrageenase Mutant

After being activated in 5 ml of a LB liquid medium (containing 50 μg/mL kanamycin), the recombinant expression strain of E. coli was inoculated into an LB medium containing kanamycin sulfate (50 μg/mL) at an inoculation dosage of 1%, and cultured at 37° C. and 200 rpm for 6 hours; and when the OD (600) value of the bacterial solution is 0.6, 1‰ IPTG (100 mM/L) was added into the bacterial solution, and low temperature induction was carried out at 20° C. for 20 hours to express the λ-carrageenase mutant.

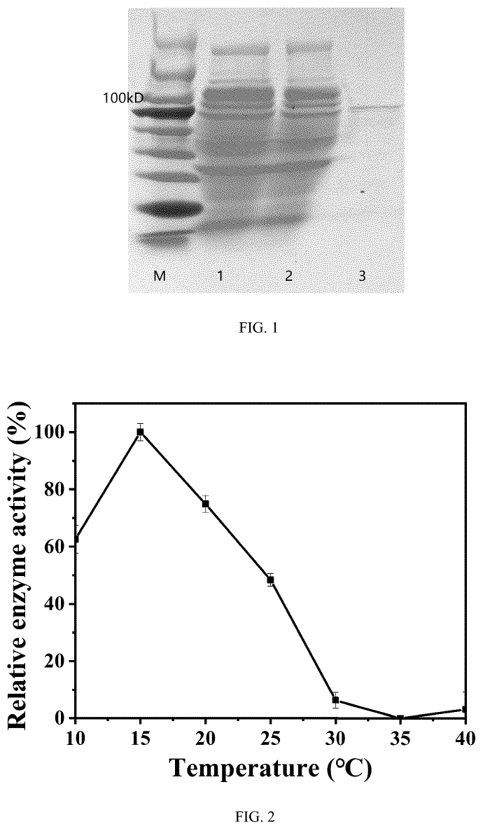

After fermentation, 8000 g of a bacterial solution was centrifuged for 10 min; bacterial cells were collected and resuspended in 50 mM of a Tirs-HCl buffer solution with pH value of 8.0, and then subjected to ultrasonic disintegration in an ice water bath for 30 min (200 W, turning on for 3 s, and turning off for 3 s); and after that, 8000 g of bacterial solution was centrifuged again for 15 min, and the supernatant i.e., a crude enzyme solution, was collected. Based on protein fused to a His tag, the crude enzyme solution was purified by affinity chromatography using Ni-NTA column, 10 mM of a low-concentration imidazole solution (10 mM of imidazole, 500 mM of NaCl, 50 mM of Tris-HCl) was used to balance the column, 20 mM of a imidazole solution (20 mM of imidazole, 500 mM of NaCl, 50 mM of Tris-HCl) was used to elute hybrid proteins with weak binding force, and 100 mM of a imidazole solution (100 mM of imidazole, 500 mM of NaCl, 50 mM of Tris-HCl) was used to elute a target protein; and buffer elution components of this part were collected to obtain a purified λ-carrageenase mutant solution (protein content is 0.5 mg/mL), and used as the enzyme solution for the study of the following Examples. SDS-PAGE was used to detect the protein purity and molecular weight (the results are shown in FIG. 1). The results show that electrophoretic pure protein with molecular weight of about 93.7 Kda can be obtained from the recombinant protein purified by the affinity column, which is consistent with the predicted results, that is, the target protein is obtained.

### Example 5 Determination of Specific Enzyme Activity of λ-Carrageenase Mutant

The standard method for determining the activity of the λ-carrageenase mutant OUC-CglA-DPQQ was as follows: in a 140 μL of reaction system containing 40 μL of an enzyme solution and 100 μL of a 3 mg/ml (m/v, mg/ml) λ-carrageenan solution dissolved in a phosphate buffer solution with pH 7, a reaction was carried out at 15° C. for 1 hour; the reaction sample was mixed with 200 μL of a DNS reagent, and the obtained mixture was boiled in a boiling water bath for 5 min for color development; and the absorbance of the product was detected at 540 n.

The enzyme activity is defined as an amount of enzyme required to produce 1 μM of reducing sugar per min under standard conditions. As determined, the activity of the λ-carrageenase mutant purified in Example 4 can reach 51.59 U/mg.

### Example 6 Determination of Optimum Reaction Conditions for λ-Carrageenase Mutant

The purified λ-carrageenase mutant obtained in Example 4 was enabled to react at different temperatures and pH to determine the effects of temperatures and pH on the enzyme activity.

The temperatures such as 10° C., 15° C., 20° C., 25° C., 30° C., 35° C. and 40° C. were selected, and a reaction was carried out for 1 hour according to the determination method in Example 5 to determine the optimum temperature.

At 15° C., the buffer solutions with pH values of 4.0-9.0 were selected as different pH determination buffer solutions for enzyme reactions; and according to the enzyme activity of the λ-carrageenase mutant, the optimum pH of the λ-carrageenase mutant was determined.

The relative enzyme activities under different conditions were calculated based on the highest enzyme activity of 100%. The results are as shown in FIG. 2 and FIG. 3. The optimum reaction temperature of the λ-carrageenase mutant is 15° C., and the optimum pH thereof is 7. The phosphate buffer solution may have adverse effects on enzyme protein activity.

### Example 7 Determination of a Degradation Product of the λ-Carrageenase Mutant

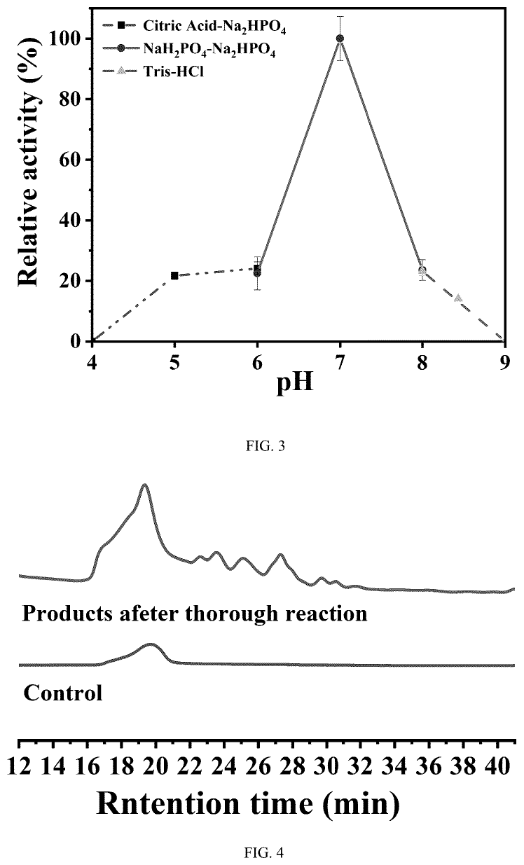

40 μL of an enzyme solution was added to 100 μL of a 3 mg/ml (m/v, mg/ml) λ-carrageenan solution dissolved in phosphate buffer solution with pH 7, a reaction was carried out at 15° C. for 12 hours, and the product was detected by a high-performance liquid chromatography. The results are as shown in FIG. 4. The results show that the product obviously contains long-chain oligosaccharides such as decasaccharide, dodecasaccharide, and tetradecasaccharide.

### Example 8 Definition of Polymerization Degree Composition of λ-Carrageenase Mutant Product

40 μL of an enzyme solution was added to 100 μL of a 3 mg/ml (m/v, mg/ml) λ-carrageenan solution dissolved in a phosphate buffer solution with pH 7, a reaction was carried out at 15° C. for 12 hours, and the product was detected by ESI-MS. The results are as shown in FIG. 5. The results show that the product contains λ-carrageenan oligosaccharides, i.e., long-chain oligosaccharides such as decasaccharide, dodecasaccharide, and tetradecasaccharide.

### Example 9 Preparation of an Enzyme Preparation

For the enzyme solution of Example 4, imidazole was replaced with a Tris-HCl buffer solution with pH 7.0, and the product was lyophilized, so that pure enzyme powder was obtained and stored.

The foregoing examples are provided to those skilled in the art to fully disclose and describe how to implement and use the claimed embodiments, and are not intended to limit the scope of the disclosure herein. Modifications apparent to those skilled in the art will fall within the scope of the appended claims.

1. A λ-carrageenase mutant OUC-CglA-DPQQ, which has an amino acid sequence as set forth in SEQ ID NO. 1.

2. An encoding gene of the λ-carrageenase mutant OUC-CglA-DPQQ of claim 1, wherein the encoding gene has a nucleotide sequence as set forth in SEQ ID NO. 2.

3. An application of the λ-carrageenase mutant OUC-CglA-DPQQ according to claim 1 in degradation of carrageenan/preparation of λ-carrageenan oligosaccharides with high polymerization degree.

4. A method for degrading carrageenan/preparing λ-carrageenan oligosaccharides with high polymerization degree, wherein the λ-carrageenase mutant OUC-CglA-DPQQ according to claim 1 is used to degrade the carrageenan to obtain the λ-carrageenan oligosaccharides with high polymerization degree.

5. The method for degrading carrageenan/preparing λ-carrageenan oligosaccharides with high polymerization degree according to claim 4, wherein the λ-carrageenan oligosaccharides with high polymerization degree comprise any one or more of decasaccharide, dodecasaccharide, tetradecasaccharide, hexadecasaccharide, octadecasaccharide and icosaccharide.

6. The method for degrading carrageenan/preparing λ-carrageenan oligosaccharides with high polymerization degree according to claim 4, wherein the degradation is carried out at 10-25° C., pH 6.0-8.0, for 1-12 hours or the carrageenan exists in the form of a solution, the concentration of λ-carrageenan is 2-5 mg/ml, and the enzyme dosage of the λ-carrageenase mutant OUC-CglA-DPQQ is 0.20-0.25 U.

7. A recombinant expression vector, which carries the encoding gene of λ-carrageenase mutant OUC-CglA-DPQQ according to claim 2.

8. A recombinant engineering bacterium, having the encoding gene of λ-carrageenase mutant OUC-CglA-DPQQ according to claim 2 inserted into a genome thereof so as to be capable of expressing the λ-carrageenase mutant OUC-CglA-DPQQ.

9. An application of the recombinant expression vector according to claim 7 in preparation of λ-carrageenase mutant OUC-CglA-DPQQ.

10. An enzyme preparation, comprising the λ-carrageenase mutant OUC-CglA-DPQQ according to claim 1.

11. An application of the recombinant engineering bacterium according to claim 8 in preparation of λ-carrageenase mutant OUC-CglA-DPQQ.


SEQUENCE LISTING (4 sequences)

SEQ ID NO: 1 - protein, 818 aa, synthetic construct
MRENNAPMYAVTVITKDKTPYIVCGGYDKNIYYLSSSGDLLKTIASKDYSVEKAKGANSKEVPTGNAHISNFIRTIKKADGSEVLAVQGVNHTMSIQARGSLYLFHPLDEKPFKTIDLEGKRLLGELKVVDVGLDGDQEIIAGSSTMIHESSLLKVDLETYEQELFEISKIKKKIDRFGYRVTQPEIITQNGKQSYFVLFGSRILLVPLDFNLEKTEVLVSKYSFNDMWKDGKGNIILASIQSGGSEIYVLNPNKPNWKQAYENLIPKGKIASIIANTKEVKEQLNVFTAPIEERKPLPVYLMSEGVPPSIKTLVDDIKENYKSPVFLNRSGSDKEKWDRSSIENEKYRDRRDRRMKYILSQDQVIKKITSKYKGGPGIAYWGGHGNDPYMYQLSTTKKVLNAAKGKKTVLIFPELEDHSDNFNYVMEDYFYPLAKHARETNANIYVRTKHAFWQANVYLPTWSRLVSGEFSDVFVPAMEETTDKSMDLSIAGRLGIWTSGATDSWGSRCARDNPSLDRLRQHSHQMLPNHFLRNMIYHVSSGAQYLDNFNVDQEYMSLLWELIAKGALYVPKRSEILSFSPVNLGMLSPDKHYLEESSNAKWTTFYDEEKEKNNPLVFSHLNGSWPGAPVTEWDFSKYAGGVQDRRLNFLPTYTNGLVLLTPPQNGIYADKKAKRGTLTSHLHPMYKNIMKEYYTDGRNYYSADGTQTYKANEYYKVIAKDIKDNAKKLPLTVSGDVAWVVAQSSPKHLRLTLIDGGYLNPDDREVTIQFNTVVPIKVTDLLSGEEFTPNANGELQIKVSCGLFRFIDIEFTGFKLE

SEQ ID NO: 2 - DNA, 2454 bp, synthetic construct
  source 1..2454: mol_type = other DNA
atgcgcgaaaacaacgcgccaatgtacgcagtaaccgttattaccaaagataaaaccccgtacattgtatgcggtggttatgacaaaaatatctactacctgtctagttccggcgatctgctgaaaaccattgcgagcaaagactattcggttgaaaaagcaaaaggcgcgaacagcaaagaagtgccgactggcaacgcccacatttccaacttcattcgcaccatcaaaaaagcagacggttctgaagtgctggcggttcagggcgtgaaccacaccatgtccatccaagctcgcggtagtctgtacctgttccatccgctggacgaaaaaccgtttaaaaccattgatctggaaggcaaacgtctgctgggcgaactgaaggtggttgatgttggcctggacggcgaccaggaaattatcgcgggttcttccaccatgatccacgaatcttctctgctgaaagttgatctggaaacctacgaacaggaactgttcgaaatcagcaaaattaaaaagaaaatcgatcgcttcggctatcgcgtgacccagccggaaatcattactcagaacggtaaacagtcttacttcgtgctgttcggcagccgtatcctgctggtgccgctggattttaacctcgaaaaaaccgaagttctggttagcaaatacagcttcaatgacatgtggaaagatggcaaaggcaatatcatcttggcgagcatccagagcggtggctctgaaatctacgttctgaacccgaacaaaccaaactggaaacaggcgtacgaaaacctgatcccgaaaggcaaaatcgcgagcattatcgccaataccaaagaagttaaagaacagctgaacgtttttaccgcgccgatcgaagaacgcaaaccgctgccggtttacctgatgtctgagggtgtgccgccgagcatcaaaacgctggttgatgatatcaaagaaaactacaaatctccggttttcctgaaccgcagcggttctgacaaagaaaaatgggatcgctcctctatcgaaaacgaaaaatatcgtgatcgccgcgaccgccgtatgaaatacattctgtctcaggatcaggtcatcaagaaaatcacatcgaaatacaaaggcggtccgggcatcgcctactggggcggtcacggcaacgatccgtacatgtaccagctgtctaccactaagaaagtgctgaacgctgcgaaaggcaagaaaactgtgctgattttcccggaattggaagatcactctgataatttcaactacgttatggaagattacttctacccgctggccaaacacgctcgtgaaaccaacgccaacatctatgttcgtaccaagcacgcgttttggcaggcaaatgtgtatttgccgacctggtcccgtctggtttcgggcgagttttctgatgtgtttgttccggcaatggaagaaaccaccgacaagagcatggacctgagcatcgcgggccgtctgggcatttggaccagcggcgctaccgactcctggggcagccgttgcgcacgtgataacccgagcctggatcgtctgcgccagcatagccaccagatgctgccgaaccacttcctgcgcaacatgatttaccatgttagctccggtgcgcagtacctggataacttcaacgtggaccaggaatacatgagcctgctgtgggaactgattgctaaaggtgcactgtacgttccgaaacgtagtgaaattctgtccttctctccggttaacctgggtatgctgagcccggataaacactacctggaggaaagcagtaacgcgaagtggaccaccttctacgatgaagaaaaagagaaaaacaaccctctggtctttagccacctgaacggttcttggccgggcgcacccgtgaccgaatgggatttttctaaatatgcgggtggcgtgcaggatcgtcgtctgaacttcctgccgacctatactaacggcctggtactgctgactccgccgcagaacggtatctacgctgataaaaaagcgaaacgtggtaccctgactagccacctgcacccgatgtacaaaaacattatgaaggaatattacaccgatggccgtaattactacagcgcggacggcacccagacctacaaagcgaatgaatactataaagtgatcgcaaaagatattaaagataacgcgaaaaaactgccgctgaccgttagcggtgacgtggcgtgggttgttgcgcagagctccccgaaacatctgcgtctgaccctgatcgacggcggctacctgaacccggacgaccgtgaagtgaccatccagttcaacaccgtggttccgattaaagttactgacctgctgagcggtgaagaattcactccgaacgctaacggtgaactgcagattaaagttagttgcggcctgttccgtttcattgacatcgaattcaccggcttcaaactcgag

SEQ ID NO: 3 - DNA, 30 bp, synthetic construct
  source 1..30: mol_type = other DNA
agatataccatgcgcgaaaacaacgcgcca

SEQ ID NO: 4 - DNA, 31 bp, synthetic construct
  source 1..31: mol_type = other DNA
gcgttgttttcgcgcatggtatatctccttc